(19) United States
(12) Patent Application Publication
(10) Pub. No.: US 2024/0188997 A1
(43) Pub. Date: Jun. 13, 2024

(54) CERVICAL LAMINOPLASTY INSTRUMENTS AND PROCEDURES

(71) Applicants: Cor Medical Ventures, Inc., Del Mar, CA (US); The Regents of the University of Colorado, a body corporate, Denver, CO (US)

(72) Inventors: Vikas Patel, Denver, CO (US); Brian Bowman, Carlsbad, CA (US); Maisha Zaman, Carlsbad, CA (US); Amber McGrogan, San Jose, CA (US)

(21) Appl. No.: 18/535,931

(22) Filed: Dec. 11, 2023

Related U.S. Application Data
(60) Provisional application No. 63/431,831, filed on Dec. 12, 2022.

(51) Int. Cl. A61B 17/70; A61B 17/80; A61F 2/46

(52) U.S. Cl. CPC A61B 17/7071; A61B 17/7044; A61B 17/7059; A61B 17/808; A61F 2/4611; A61F 2002/4625

(57) ABSTRACT

Devices and methods for performing a cervical laminoplasty procedure on a patient. An exemplary device includes a primary instrument having a proximal portion and a distal portion, a secondary instrument having a distal portion, and an implant plate assembly having an implant plate and an implant post. The distal portion of the primary instrument can be configured to engage the implant post of the implant plate assembly. A lateral side of the implant plate can engage a lateral portion of a lamina of a vertebra, and a medial side of the implant plate can engage a medial portion of a lamina of a vertebra. The implant plate can be bent and a corresponding adjustment in an angle between the lateral and medial portions of the lamina can be achieved.

### CROSS-REFERENCES TO RELATED APPLICATIONS

This application claims the benefit of U.S. Provisional Patent Application No. 63/431,831 filed Dec. 12, 2022, the content of which is incorporated herein by reference.

### BACKGROUND

Embodiments of the present invention relate generally to surgical systems and methods, and in particular, to cervical laminoplasty instruments and procedures.

Surgeons may follow any of a variety of procedures in order to perform cervical laminoplasty. Standard cervical laminoplasty procedures are similar in that they expand the cervical canal; generally, by hinging open the compressed vertebra. In Hirabayashi (open door) laminoplasty, a trough is drilled all the way through the laminae on one side, and partially through the laminae on the other. The lamina is gently rotated towards the “closed side” to relieve pressure on the spinal nerves. There are various known ways of keeping the vertebra lifted off the spinal nerves including titanium mini plates, bone grafts, and ceramic spacers. French door laminoplasty is used in order to create a symmetric expansion of the canal. Instead of creating a through trough on one side and a partial trough on the other side of the vertebra, French door laminoplasty creates two partial troughs on either side of the vertebra and a cut down the middle. This allows both pieces of the vertebra to be rotated toward the lateral of the body and relieve pressure on the spinal nerves. Although existing procedures can provide beneficial results for patients, still further improvements are desired. Embodiments of the present invention provide solutions to at least some of these outstanding needs.

### SUMMARY

The present disclosure generally relates to medical devices and processes and more particularly relates to cervical laminoplasty instruments and procedures.

In exemplary embodiments, methods can include drilling bone to create a hinge. In some cases, a plate can be placed before making a contralateral cut through the lamina. This can facilitate placement of a plate and screws. After placing a plate and screws, the surgeon can then cut through the contralateral lamina and finally bend the plate.

In exemplary embodiments disclosed herein, a surgeon can perform cervical laminoplasty without drilling bone to create a hinge. For example, in some cases a surgeon can create a hinge by bending a plate to a desired degree.

Advantageously, these approaches can avoid certain complications which may be associated with the Hirabayashi and/or French door procedures. For example, in regard to the partial troughs required by some existing methods, the surgeon may need to go back and forth between the drill and the vertebra to test for adequate bone hinging. Embodiments of the present invention can avoid such requirements. Relatedly, embodiments of the present invention can avoid problems associated with some current methods, such as the risk of removing too much of the bone to create the hinge.

In one aspect, embodiments of the present invention encompass cervical laminoplasty systems having a primary instrument, a secondary instrument, and an implant plate. In some cases, a primary instrument includes a proximal portion and a distal portion. In some cases, an implant plate includes a medial side and a lateral side. Cervical laminoplasty systems can also include one or more medial bone screws configured to secure a medial side of the implant plate to a medial portion of a lamina of a vertebra. and one or more lateral bone screws configured to secure a lateral side of the implant plate to a lateral portion of a lamina of a vertebra. In some cases, the proximal portion of the primary instrument includes a handle. In some cases, the distal portion of the primary instrument is configured to engage the implant. In some cases, the distal portion of the primary instrument is configured to engage an implant post of the implant plate assembly. In some cases, the implant plate includes an implant post, and the primary instrument is configured to engage the implant post. In some cases, a cervical laminoplasty system further includes a secondary instrument having a proximal portion and a distal portion, where the proximal portion is configured to engage the primary instrument, and where the distal portion is configured to engage a second lamina of the vertebra. In some cases, the primary instrument and the secondary instrument are coupled together via a screw. In some cases, the implant plate of the implant plate assembly includes a lateral side, a medial side, and a bridge disposed between the lateral side and the medial side. In another aspect, embodiments of the present invention encompass methods of performing a cervical laminoplasty procedure. Exemplary methods can include creating a partial cut in a first lamina of a vertebra of a patient, and attaching an implant plate with the vertebra of the patient. A lateral side of the plate can be attached with a lateral portion of the first lamina of the vertebra, and a medial side of the plate can be attached with a medial portion of the first lamina of the vertebra. Methods can also include creating a full cut in a second lamina of the vertebra of the patient, engaging a primary instrument of a cervical laminoplasty system with the implant plate, and moving the first instrument of the cervical laminoplasty system relative to the patient to form a bend in the implant plate. The bend can be disposed between the lateral side of the implant plate and the medial side of the implant plate. In some cases, the primary instrument includes a distal portion and the secondary instrument includes a distal portion, the step of engaging the primary instrument of the cervical laminoplasty system with the implant plate includes engaging the distal portion of the primary instrument with the medial side of the implant plate, and the step of engaging the secondary instrument of the cervical laminoplasty system with the full cut includes engaging the distal portion of the secondary instrument with the full cut. In some cases, the medial side of the implant plate includes an implant post, and the step of engaging the distal portion of the primary instrument with the medial side of the implant plate includes engaging the distal portion of the primary instrument with the implant post of the implant plate. In some cases, the distal portion of the primary instrument includes a hook, and the step of engaging the distal portion of the primary instrument with the implant post includes engaging the hook of the distal portion of the primary instrument with the implant post. In some cases, the step of attaching the implant plate with the vertebra of the patient includes securing the lateral side of the implant plate with the lateral portion of the first lamina using one or more lateral bone screws. In some cases, the step of attaching the implant plate with the vertebra of the patient includes securing the medial side of the implant plate with the medial portion of the first lamina using one or more medial bone screws. In some cases, the implant plate includes a bridge disposed between the lateral side of the implant plate and the medical side of the implant plate, and the step of forming the bend in the implant plate includes bending the bridge. In some cases, the bend has an angle of about 60 degrees. In some cases, the bend has an angle with a value within a range from about 1 degree to about 60 degrees. In some cases, the step of moving the first instrument and the second instrument of the cervical laminoplasty system relative to the patient includes rotating the first instrument and the second instrument of the cervical laminoplasty system relative to the lateral portion of the first lamina of the vertebra.

In another aspect, embodiments of the present invention encompass cervical laminoplasty systems that include a primary instrument having a proximal portion and a distal portion, a secondary instrument having a distal portion, and an implant plate assembly having an implant plate and an implant post. In some cases, the proximal portion of the primary instrument includes a handle. In some cases, the distal portion of the primary instrument includes a hook. In some cases, the distal portion of the primary instrument is configured to engage the implant post of the implant plate assembly. In some cases, the primary instrument and the secondary instrument are coupled together via a wing screw or other attachment means. In some cases, the implant plate of the implant plate assembly includes a lateral side configured to engage a lateral portion of a lamina of a vertebra. In some cases, the implant plate of the implant plate assembly includes a medial side configured to engage a medial portion of a lamina of a vertebra. In some cases, the system further includes one or more lateral bone screws configured to secure a lateral side of the implant plate to a lateral portion of a lamina of a vertebra. In some cases, the system further includes one or more medial bone screws configured to secure a medial side of the implant plate to a medial portion of a lamina of a vertebra. In some cases, the implant plate of the implant plate assembly includes a lateral side, a medial side, and a bridge disposed between the lateral side and the medial side. In yet another aspect, embodiments of the present invention encompass method of performing a cervical laminoplasty procedure. An exemplary method can include creating a partial cut in a first lamina of a vertebra of a patient, attaching an implant plate with the vertebra of the patient, where a lateral side of the plate is attached with a lateral portion of the first lamina of the vertebra and a medial side of the plate is attached with a medial portion of the first lamina of the vertebra. Methods can also include creating a full cut in the second lamina of the vertebra of the patient, engaging a primary instrument of a cervical laminoplasty system with the implant plate, engaging a secondary instrument of the cervical laminoplasty system with the full cut, and moving the first instrument and the second instrument of the cervical laminoplasty system relative to the patient to form a bend in the implant plate, where the bend is disposed between the lateral side of the implant plate and the medical side of the implant plate. In some cases, the primary instrument includes a distal portion and the secondary instrument comprises a distal portion, and the step of engaging the primary instrument of the cervical laminoplasty system with the implant plate includes engaging the distal portion of the primary instrument with the medial side of the implant plate, and the step of engaging the secondary instrument of the cervical laminoplasty system with the full cut includes engaging the distal portion of the secondary instrument with the full cut. In some cases, the medial side of the implant plate includes an implant post, and the step of engaging the distal portion of the primary instrument with the medial side of the implant plate includes engaging the distal portion of the primary instrument with the implant post of the implant plate. In some cases, the distal portion of the primary instrument includes a hook, and the step of engaging the distal portion of the primary instrument with the implant post includes engaging the hook of the distal portion of the primary instrument with the implant post. In some cases, the step of attaching the implant plate with the vertebra of the patient includes securing the lateral side of the implant plate with the lateral portion of the first lamina using one or more lateral bone screws. In some cases, the step of attaching the implant plate with the vertebra of the patient includes securing the medial side of the implant plate with the medial portion of the first lamina using one or more medial bone screws. In some cases, the implant plate includes a bridge disposed between the lateral side of the implant plate and the medical side of the implant plate, and the step of forming the bend in the implant plate includes bending the bridge. In some cases, the bend has an angle of about 60 degrees. In some cases, the bend has an angle with a value within a range from about 1 degree to about 60 degrees. In some cases, the step of moving the first instrument and the second instrument of the cervical laminoplasty system relative to the patient includes rotating the first instrument and the second instrument of the cervical laminoplasty system relative to the lateral portion of the first lamina of the vertebra.

Additional aspects of embodiments of the invention will be apparent from the detailed descriptions and claims herein.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B illustrate aspects of a cervical laminoplasty instrument assembly, in accordance with some embodiments;

FIGS. 2A and 2B illustrate aspects of a cervical laminoplasty instrument and plate assembly, in accordance with some embodiments;

FIGS. 3A and 3B illustrate aspects of a cervical laminoplasty instrument and plate assembly, in accordance with some embodiments;

FIGS. 4A and 4B illustrate aspects of a cervical laminoplasty plate assembly, in accordance with some embodiments;

FIGS. 5A and 5B illustrate aspects of a cervical laminoplasty plate assembly, in accordance with some embodiments;

FIGS. 6A and 6B illustrate aspects of a cervical laminoplasty instrument and plate assembly, in accordance with some embodiments;

FIGS. 7A and 7B depict aspects of a medial bone screw, in accordance with some embodiments;

FIGS. 8A and 8B depict aspects of a lateral bone screw, in accordance with some embodiments;

FIGS. 9A and 9B depict aspects of a post screw, in accordance with some embodiments;

FIGS. 10A to 10F depict aspects of a cervical laminoplasty procedure, in accordance with some embodiments;

FIG. 11 illustrates aspects of a cervical laminoplasty instrument assembly, in accordance with some embodiments;

FIG. 12 illustrates aspects of a cervical laminoplasty instrument assembly, in accordance with some embodiments;

FIG. 13 depicts aspects of a cervical laminoplasty procedure, in accordance with some embodiments;

FIG. 14 depicts aspects of a cervical laminoplasty procedure, in accordance with some embodiments;

FIGS. 15A to 15G depict aspects of a cervical laminoplasty plate assembly, in accordance with some embodiments;

FIG. 16 depicts aspects of a cervical laminoplasty instrument and plate assembly, in accordance with some embodiments; and

FIG. 17 depicts aspects of a cervical laminoplasty instrument and plate assembly, in accordance with some embodiments.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

An exemplary cervical laminoplasty system includes an implant plate, a post screw, medial and lateral clover bone screws, an instrument assembly, and involves a procedure of operation. The implant plate, which can be configured to lay across a single cervical vertebra, can include medial and lateral sides connected by a thin bridge. In some cases, the post screw is threaded only part way up the main shaft in order to be secured to the plate. The post screw can then used mid-procedure in order to force a bend in the plate across its thin bridge; hinging the vertebra open. In some cases, the medial and lateral clover bone screws are self-tapping, and inserted directly into the vertebra in order to secure the plate in place. In some cases, a clover designation of the bone screws is derived from the Torx® head of the screws, which can be set at a standard size to allow the surgeon ease in procuring their own driver.

In some cases, the primary instrument resembles a miniature prybar. It can include a handle on one end and a horizontal hook which fits to the base of the spherically headed post screw on the other. A secondary instrument which attaches by a wing screw or other attachment means to the shaft of the primary instrument can extend a similar length as the primary instrument. This secondary instrument can be designed to hook to the end of the severed vertebra and encourage a hinge in the implant plate by the surgeon's rotation of the handle.

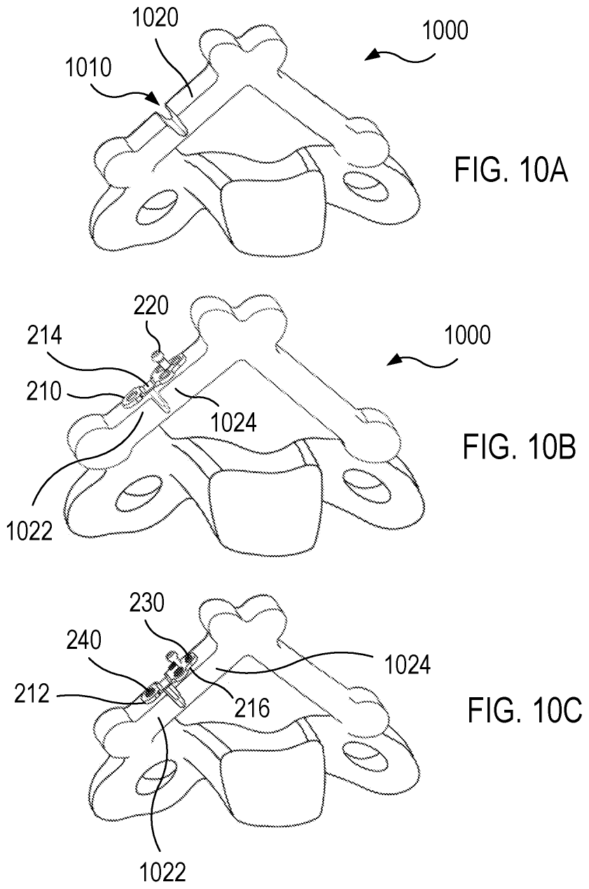

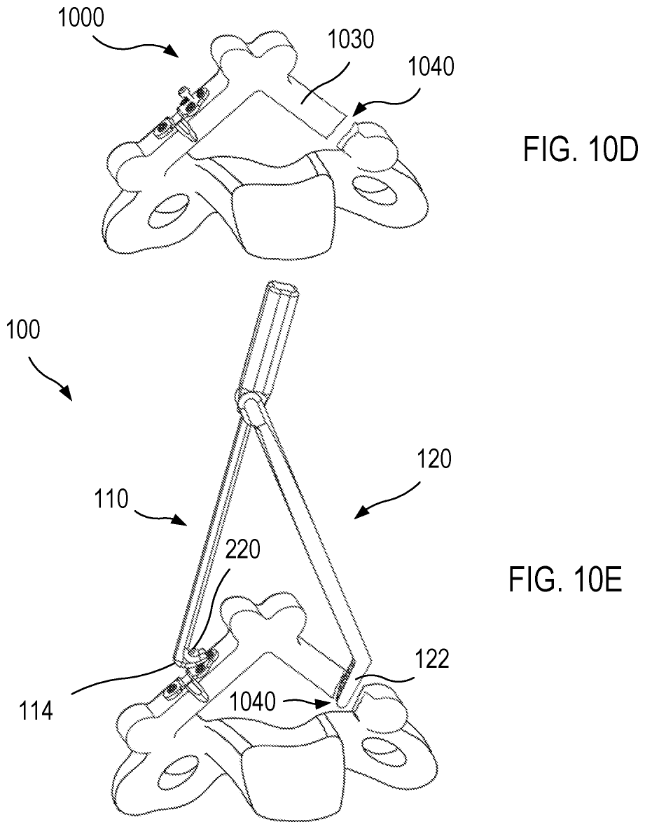

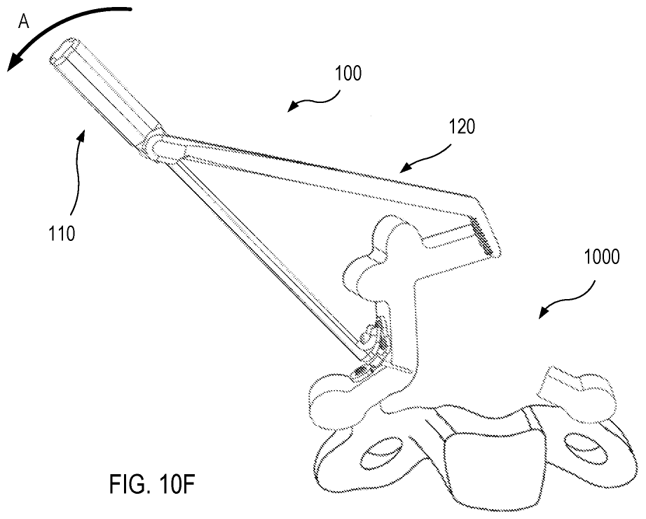

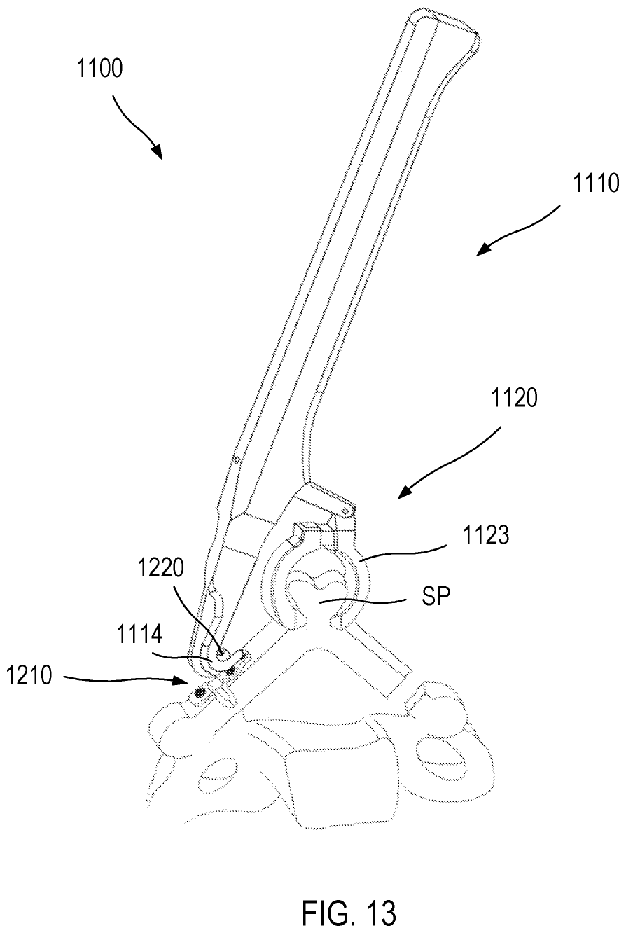

A secondary instrument can be configured in a variety of ways, and in use can operate to aid the primary instrument in hinging open a vertebra of the patient. For example, as shown in FIG. 10E, a secondary instrument 120 can operate as a hook to reach the end of a severed bone. Likewise, as shown in FIG. 13, a secondary instrument 1120 can operate as a clamp for the spinous process. Either embodiment can be used to aid a primary instrument in its purpose of hinging the vertebra open.

In some cases, method embodiments involve administering general anesthesia to the patient and positioning them to give the surgeon clear access to the rear of their neck. An incision ranging from three to five inches can be made in order to expose the lamina of the vertebra. A partial cut can be made on one side of the lamina to allow the bone to hinge open. With the post screw already threaded into it, the plate can be secured over this cut with the medial and lateral clover bone screws. The bridge of the plate can lay over the cavity created. A full cut can then be made all the way through the opposing lamina. Then, hooking the primary instrument to the large post screw and the secondary instrument to the end of the severed bone (or clamping onto the spinous process), the surgeon can rotate the instrument assembly toward the lateral side of the body and force a bend in the plate in order to hinge the vertebra open. The instrument and post screw can then be removed from the plate, and the lateral and medial screws can be tightened (e.g. to their maximum). This may complete the procedure, and the patient may then be sewn up.

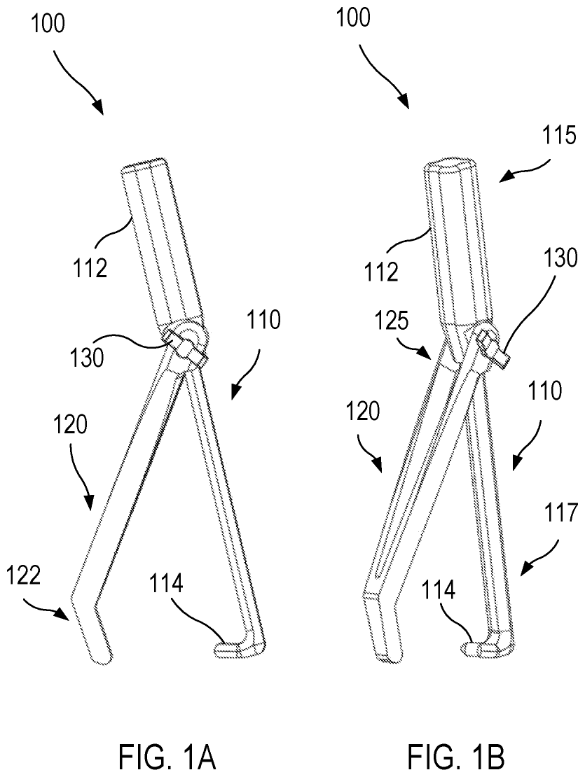

Turning now to drawings FIGS. 1A and 1B, these illustrations depict aspects of an instrument assembly 100, according to embodiments of the present invention. As shown here, an instrument assembly 100 can include a primary instrument 110 and a secondary instrument 120. The primary and secondary instruments can be coupled together. The primary instrument 110 can include a handle 112 at a proximal portion 115 of the instrument 110 and a hook 114 at a distal portion 117 of the instrument 110. The secondary instrument 120 can include a proximal portion 125 and a distal portion 122. As shown here, proximal portion 125 is coupled with or engaged with the primary instrument 110. The instrument assembly 100 can also include a wing screw 130. The wing screw 130 can operate to hold the primary and secondary instruments together. In some cases, a length of the primary instrument 110 can have a value within a range from about 60 mm to about 140 mm. In some cases, the primary and secondary instruments can be two separate entities (e.g. not attached to the primary instrument).

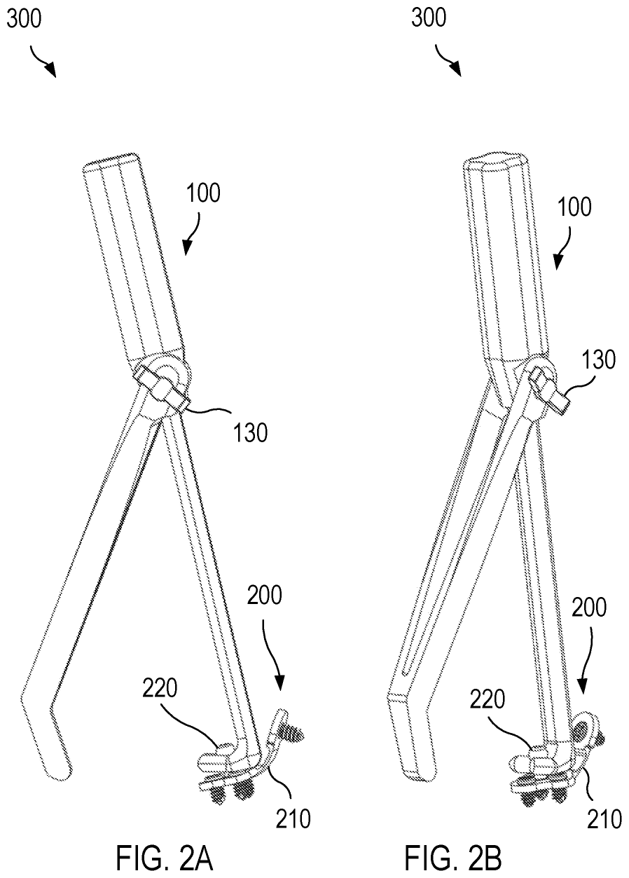

FIGS. 2A and 2B depict aspects of an instrument and plate assembly 300, according to embodiments of the present invention. As shown here, an instrument and plate assembly or cervical laminoplasty system 300 can include an instrument assembly 100 and a plate assembly 200. The instrument assembly 100 and the plate assembly 200 can be coupled together. The plate assembly 200 can include an implant plate 210 and post screw or implant post 220. In the embodiment depicted here, the implant plate 210 is shown in a bent configuration (e.g. after bending).

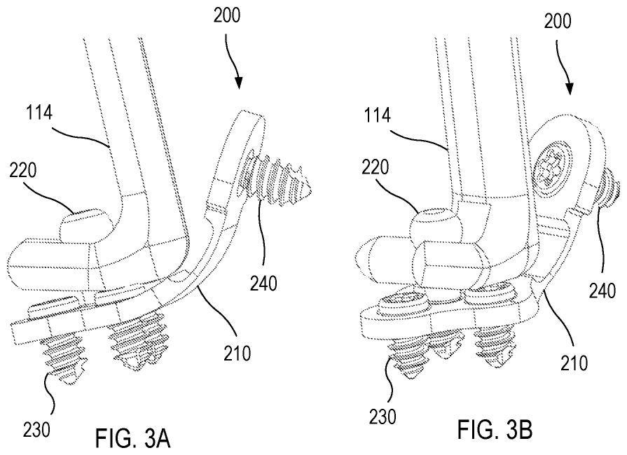

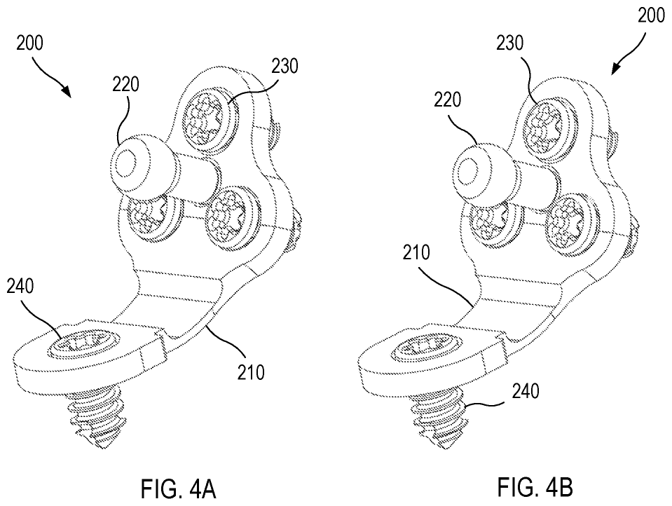

As shown in FIGS. 3A and 3B, a plate assembly 200 can also include one or more medial clover bone screws 230 and one or more lateral clover bone screws 240. Similarly, FIGS. 4A and 4B illustrate that a plate assembly 200 can include one or more medial clover bone screws 230 and one or more lateral clover bone screws 240.

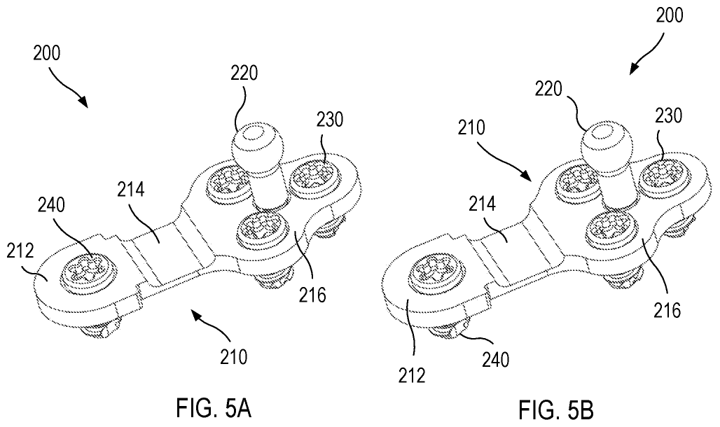

FIGS. 5A and 5B depict an implant plate assembly 200 in an unbent configuration (e.g. prior to bending). As shown here the plate assembly 200 can include an implant plate 210, a post screw 220, one or more medial clover bone screws 230, and one or more lateral clover bone screws 240. The implant plate 210 can include a lateral side 212, a bridge 214, and a medial side 216. In some cases, a length of the implant plate can have a value within a range from about 15 mm to about 25 mm in length when flat. In some cases, a width of the implant plate can have a value that is about 12 mm or less. In some cases, a length of a lateral side of the plate can have a value within a range from about 5 mm to about 7 mm. In some cases, a width of a lateral side of the plate can have a value within a range from about 4 mm to about 8 mm. In some cases, a length of a medial side of the plate can have a value within a range from about 8 mm to about 12 mm. In some cases, a width of a medial side of the plate can have a value within a range from about 8 mm to about 12 mm. In some cases, a length of the bridge of the plate can have a value within a range from about 3 mm to about 4 mm. In some cases, a width of the bridge of the plate can have a value that is about 5 mm. In some cases, a depth of the bridge of the plate can have a value within a range from about 1 mm to about 1.2 mm. In some cases, the implant plate can be configured to accommodate or receive a post screw having a diameter that is about 1.9 mm. In some cases, the implant plate can be configured to accommodate or receive a one or more medial clover screws each having a diameter that is about 2.3 mm. In some cases, the implant plate can be configured to accommodate or receive a lateral clover screw having a diameter that is about 2.5 mm.

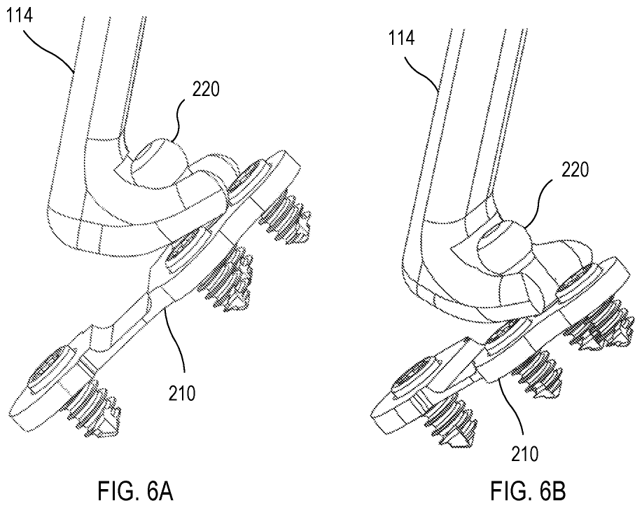

FIGS. 6A and 6B depict an operative association between the hook 114 of a primary instrument, and a post screw 220 and an implant plate 210, where the implant plate is in a flat or unbent configuration.

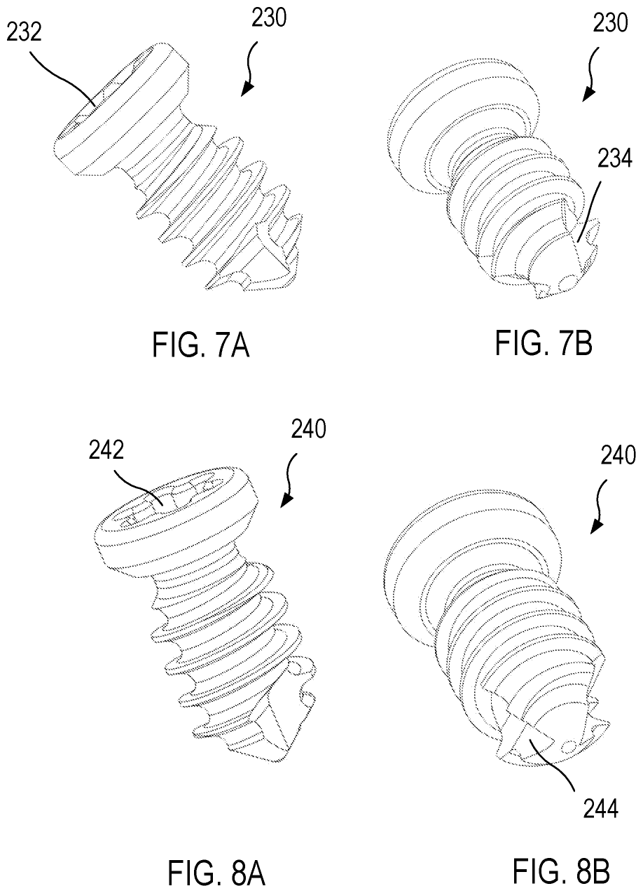

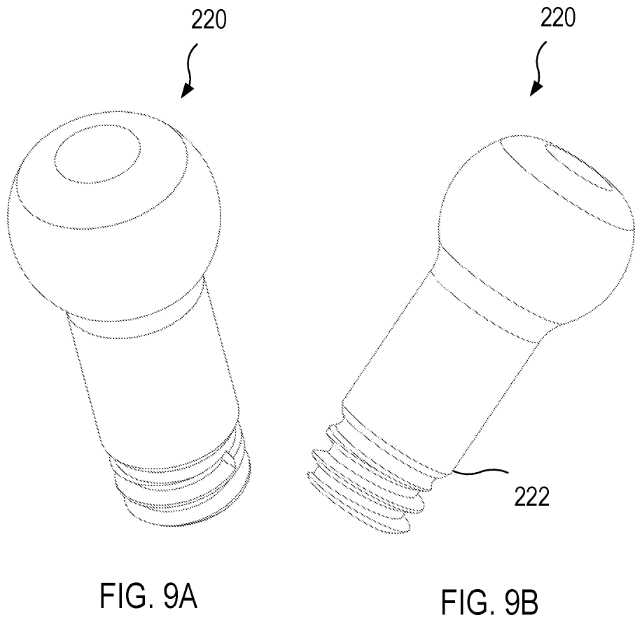

FIGS. 7A and 7B depict aspects of a medial bone screw 230, according to embodiments of the present invention. As shown here, a medial bone screw 230 can include a clover head 232 and a self-tapping flute 234. In some cases, a diameter of a head of a medial screw has a value within a range from about 2.6 mm to about 3.0 mm. FIGS. 8A and 8B depict aspects of a lateral bone screw 240, according to embodiments of the present invention. As shown here, a lateral bone screw 240 can include a clover head 242 and a self-tapping flute 244. In some cases, a diameter of a head of a lateral screw has a value within a range from about 2.9 mm to about 3.1 mm. FIGS. 9A and 9B depict aspects of a post screw 220, according to embodiments of the present invention. As shown here, a post screw 220 can include a partially threaded shaft 222. In some cases, a diameter of a head of a post screw has a value within a range from about 2.6 mm to about 3.0 mm.

FIGS. 10A to 10F depict aspects of a cervical laminoplasty procedure, according to embodiments of the present invention. As shown in FIG. 10A, one step of the procedure can include creating a partial cut 1010 in the lamina 1020 of a vertebra 1000. As shown in FIG. 10B, one step of the procedure can include placing a plate 210, with the post screw 220 already threaded therein, onto the vertebral bone, so that the bridge 214 of the plate is positioned over the partial cut or cavity. In some cases, an implant plate of the implant plate assembly includes a lateral side configured to engage a lateral portion 1022 of a lamina of a vertebra. In some cases, an implant plate of the implant plate assembly includes a medial side configured to engage a medial portion 1024 of a lamina of a vertebra. As shown in FIG. 10C, one step of the procedure can include securing the plate 210 to the lamina by placing bone screws (e.g. one or more medial bone screws 230 and one or more lateral bone screws 240) through the plate 210 and into the bone tissue. In some embodiments, one or more medial bone screws 230 can operate to secure a medial side 216 of the implant plate to a medial portion 1024 of a lamina of a vertebra 1000, and one or more lateral bone screws 240 can operate to secure a lateral side 212 of the implant plate to a lateral portion 1022 of the lamina of the vertebra 1000. As shown in FIG. 10D, one step of the procedure can include creating a full cut 1040 in the opposing side or lamina 1030 of the vertebra 1000. In some embodiments, lamina 1020 may be referred to as a first lamina and lamina 103 may be referred to as a second lamina.

As shown in FIG. 10E, one step of the procedure can include placing or positioning an instrument assembly 100 so that the hook 114 of the primary instrument 110 engages the post screw 220 (e.g. by hooking onto the post screw) and a distal portion 122 of the secondary instrument 120 engages or hooks onto the broken bone. In some cases, at least a portion of the distal portion 122 is positioned or placed in the cut 1040. As shown in FIG. 10F, one step of the procedure can include rotating or moving the instrument assembly 100 laterally (e.g. in the direction indicated by arrow A) in order to hinge open the vertebra 1000. In the embodiment depicted here, this movement of the instrument assembly 100 produces an approximately 60 degree bend in the plate (e.g. at the bridge of the plate).

According to some embodiments, a method of performing a cervical laminoplasty procedure may involve creating a partial cut 1010 in a first lamina 1020 of a vertebra 1000 of a patient as shown in FIG. 10A, and attaching an implant plate 210 with the vertebra 1000 of the patient as shown in FIG. 10B. A lateral side 212 of the plate can be attached with a lateral portion 1022 of the first lamina of the vertebra a medial side 216 of the plate can be attached with a medial portion 1024 of the first lamina of the vertebra as shown in FIG. 10C. A method can also include creating a full cut 1040 in a second lamina 1030 of the vertebra of the patient as shown in FIG. 10D. A method can further include engaging a primary instrument 110 of a cervical laminoplasty system 100 with the implant plate, and moving the first instrument 110 of the cervical laminoplasty system 100 relative to the patient to form a bend in the implant plate, as shown in FIG. 10F. The bend can be disposed between the lateral side of the implant plate and the medial side of the implant plate.

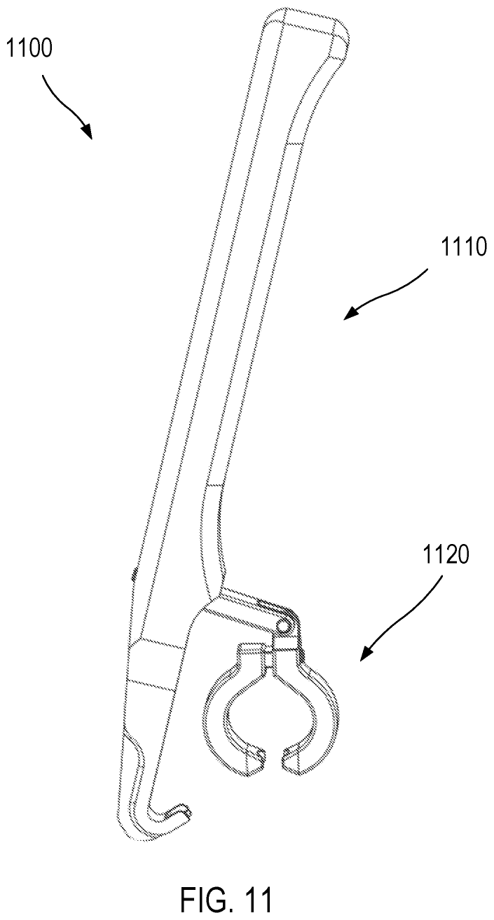

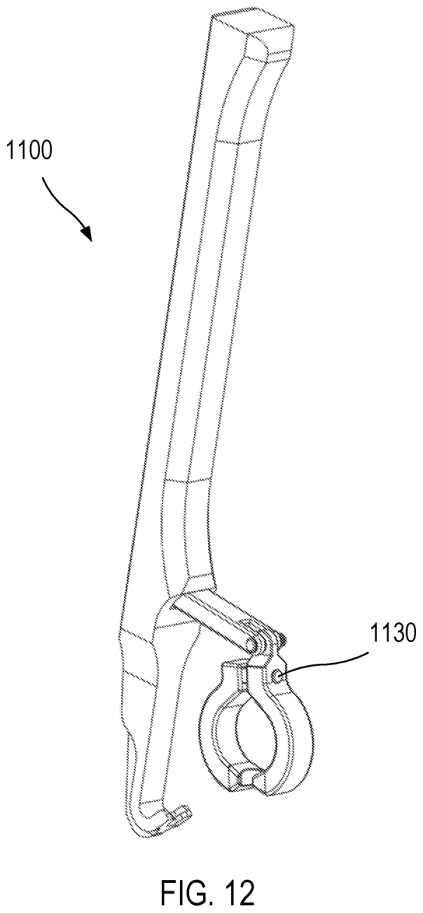

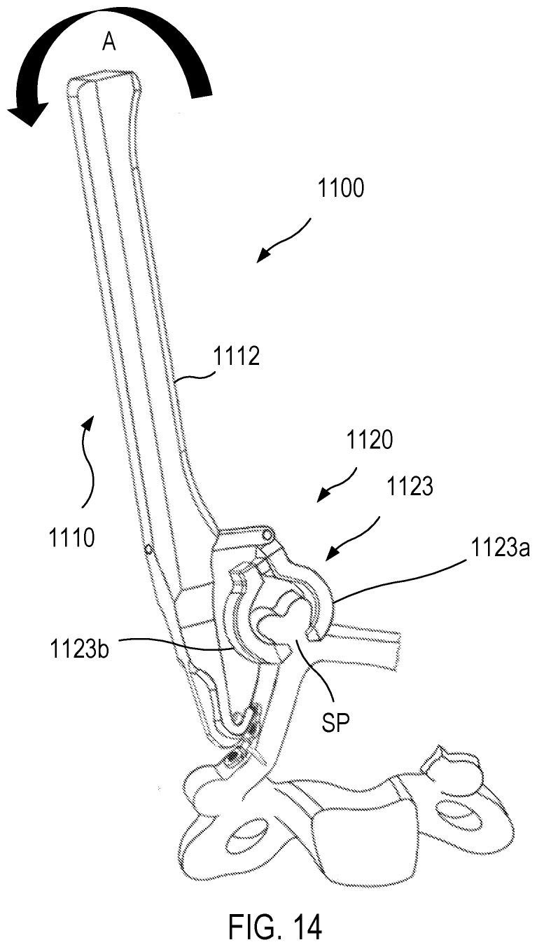

FIG. 11 depicts aspects of an instrument assembly 1100, according to embodiments of the present invention. As shown here, an instrument assembly 1100 can include a primary instrument 1110 and a secondary instrument 1120. As shown in FIG. 12, the instrument assembly 1100 can also include a fixation screw or element 1130. The fixation screw 1130 can operate to hold the primary and secondary instruments together. As shown in FIG. 13, a method can include positioning the instrument assembly 1100 so that a hook 1114 of the primary instrument 1110 hooks onto a post screw 1220 of a plate assembly 1200 and a clamp mechanism 1123 of the secondary instrument 1120 clamps onto the spinous process SP of the vertebra. The method can also include adjusting the fixation screw to tighten the clamp mechanism 1123 around the spinous process SP of the vertebrae bone. As shown in FIG. 14, a method can include rotating the instrument assembly 1100 laterally (e.g. as indicated by arrow A) so as to hinge open the vertebra. Hence, it can be seen that secondary instrument 1120 can operate as a clamp that extends from the base of the handle 1112 on the primary instrument (or is otherwise coupled with the primary instrument 1110) and is fixed to the spinous process SP of the vertebra. A clamp mechanism 1123 of the secondary instrument 1120 can be operated by adjusting a fixation screw (not shown) which forces two halves 1123a, 1123b of the clamp mechanism 1123 closer together or further apart. This secondary instrument 1120 can also aid in the hinging of the vertebra once the primary instrument 1110 begins its rotation.

Cervical laminoplasty as disclosed herein is an effective procedure for patients who have stress on their spinal nerves due to spinal stenosis. Spinal stenosis may lead to numbness, weakness, or pain in the neck and pain in shoulders, arm, or hands. Cervical laminoplasty restructures the cervical bones in order to relieve the stress on their spinal nerves. By severing one side of the vertebra and placing the implant plate on the other, the surgeon is able to put a bend in the plate; hinging the vertebra open on the severed side. This opening in the vertebra removes the stress on the spinal nerves. By using the implant plate as a hinge, the surgeon is able to open the vertebra to a specific, desired angle. This angle of the plate may have a value within a range from about 1 degree to about 60 degrees and can change dramatically from patient to patient based upon their size and amount of pressure on their spinal nerves.

As disclosed herein, exemplary systems and procedures involve an implant plate, a post screw, a primary instrument, and one or more bone screws (e.g. clover bone screws). The implant plate and bone screws can be used to secure the hinged vertebrae together. The primary instrument and post screw can operate to create the bend in the plate after the plate is attached to the vertebra and relieve the stress on the spinal nerves. As described herein, prior to these steps the procedure of severing one side of the vertebra can be performed.

In some embodiments, the plate and/or instruments can come in varying sizes depending on the patient. In some procedure embodiments, the plate can be secured to one side of the vertebra prior to the complete severing of the other side of the vertebra. In some embodiments, the number and location of the bone screws can be altered. In some cases, one or more additional screws may be added to the lateral side of the plate while up to two may be removed from the medial. In some cases, the shape of the implant plate can be altered. In many embodiments, the plate will include holes that are configured to receive the bone screws, a hole for the securement of the post screw, and a thin strip of material (e.g. bridge) to allow for a bend in the plate. In some cases, a lateral mass screw may be used instead of the lateral clover bone screws. In some cases, a lateral mass screw can operate to create additional stability when there are multiple vertebra cuts in a row as it provides a hole for a rod to slide through. In some cases, such a rod may be a standard off-the-shelf rod. In some cases, a rod can extend the length of all the hinged vertebra in order to join them together. In some cases, a lateral mass screw can be threaded into the bone early in the procedure at the same time the lateral clover bone screw would be secured in the bone. In some cases, the rod is inserted into the hole of each lateral mass screws as a final act of the procedure in order to align all hinged vertebrae together. In some cases, an additional plate can be added to the severed side of the vertebra. In such cases, this can mean that there is one hinging plate, and another reattaching the severed vertebra.

In some cases, an implant plate can be manufactured from titanium. Optionally, other biocompatible metals may be used in the manufacture of a plate. In some case, the screw components are manufactured from stainless steel or titanium. Optionally, other biocompatible metals may be used in the manufacture of the screw components. In some cases, the instrument components are manufactured from stainless steel or medical grade plastics.

Advantageously, cervical laminoplasty system and method embodiments disclosed herein can involve the use of an implant plate as a hinge to reduce risk from what is canonically done, as they do not rely on creating a trough in cervical vertebra to act as the hinge. Instead, the surgeon is able to bend an anchored metal plate to an optimal angle for the individual patient by using the primary instrument to put pressure on the post screw. The secondary instrument allows the surgeon to create the hinge with less force as it allows for equal moments to be placed on the implant plate by the primary instrument as well as the severed vertebra by the secondary instrument. Exemplary cervical laminoplasty system and method embodiments disclosed herein can involve the use of an implant plate as the hinge. In some cases, the plate is flat when initially attached, and is able to act as a hinge due to the thin or relatively more malleable layer of material connecting the medial and lateral sides of the plate and the post screw that hooks onto the primary instrument. In some cases, the plate is permanently attached with clover bone screws. By leaving one side of the vertebra severed in exemplary cervical laminoplasty systems and methods disclosed herein, there is continued relief of stress on the spinal nerves.

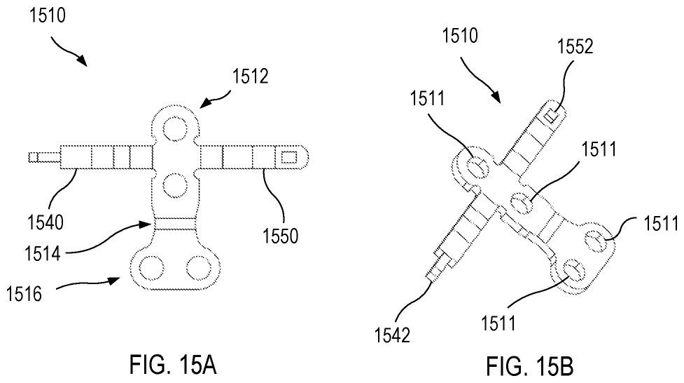

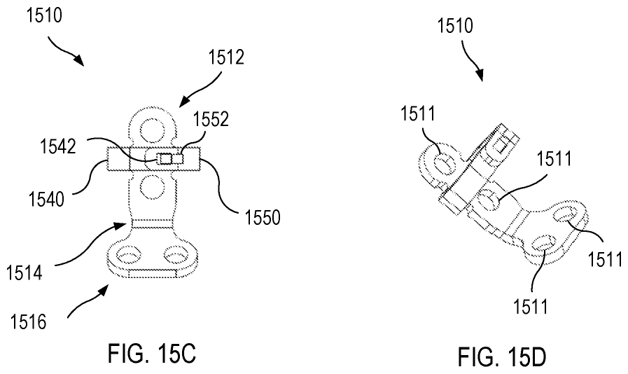

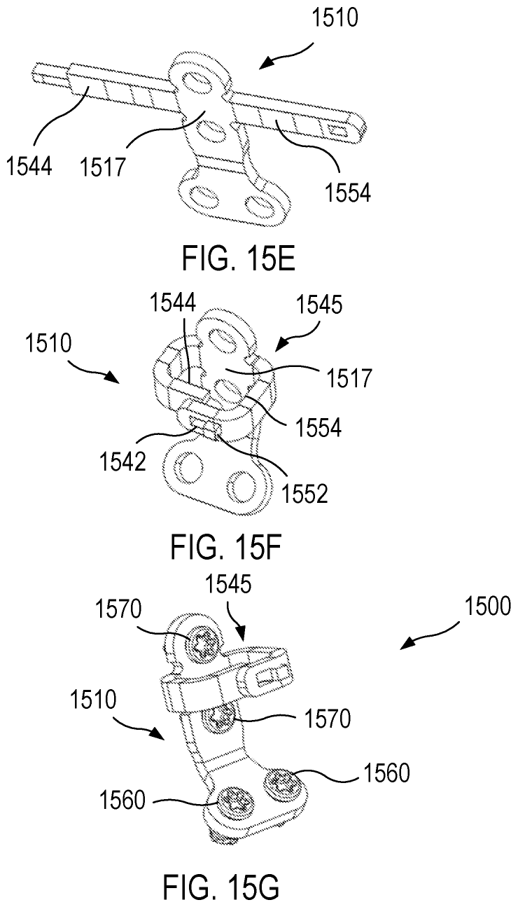

FIGS. 15A to 15G depicts aspects of an implant plate 1510, according to embodiments of the present invention. As shown in FIGS. 15A and 15B, implant plate 1510 can include a lateral side 1512, a bridge 1514, and a medial side 1516. Implant plate 1510 can also include one or more holes or apertures 1511 configured to receive and/or engage one or more respective bone screws or fixation mechanisms. Implant plate 1510 can include a first elongate member 1540 and a second elongate member 1550. According to some embodiments, implant plate 1510 can be manufactured from a flat sheet of material (e.g. metal).

As shown in FIGS. 15C and 15D, first and second elongate members 1540, 1550 can be deformed or bent (e.g. toward each other) so as to create a loop or partial loop, which in turn can be configured to engage or capture a bender mechanism. First elongate member 1540 can include a pin 1542 and second elongate member 1550 can include a slot or aperture 1552. In use, pin 1542 and slot 1552 can engage when first and second elongate members 1540, 1550 form a loop. As also shown in FIGS. 15C and 15D, implant plate 1510 can be bent or deformed at bridge 1514 (which can operate as a hinge) and thus the relative angular orientations of lateral side 1512 and medial side 1516 can be adjusted or changed.

As depicted in FIGS. 15E and 15F, either or both of the elongate arms 1540, 1550 implant plate can have an interior surface 1544, 1544 which operates to engage to contact a bender mechanism, for example after elongate arms 1540, 1550 are interdigitated with each other. As discussed elsewhere herein, a bender mechanism can have a shaft portion that is sized or configured to fit in the loop or ring 1545 created by the elongate arms (e.g. between a central inner portion 1517 of the plate 1510 and interior surface 1544.

FIG. 15G illustrates aspects of a plate assembly 1500 that includes an implant plate 1510 and one or more bone screws (e.g. lateral bone screws 1560 and medial bone screws 1570), according to embodiments of the present invention. Additional aspects of exemplary medial bone screws are discussed elsewhere herein, for example in reference to those shown in FIGS. 7A and 7B. Additional aspects of exemplary lateral bone screws are discussed elsewhere herein, for example in reference to those shown in FIGS. 8A and 8B.

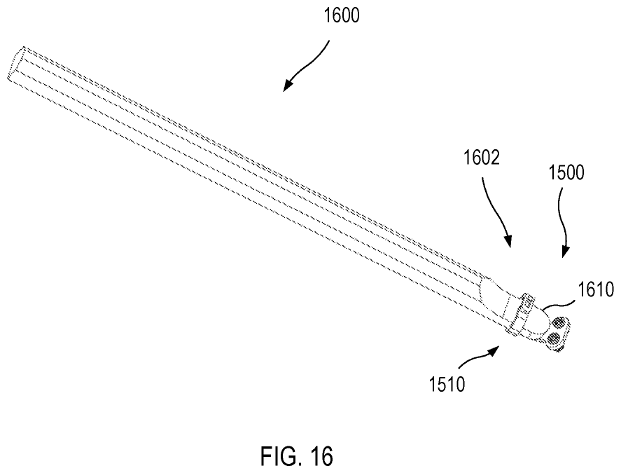

FIG. 16 depicts a bender mechanism 1600 engaged with a plate assembly 1500. Bender mechanism 1600 includes a distal section 102 having a shaft portion 1610 that is sized or configured to fit in the loop or ring created by elongate arms of an implant plate 1510 of the plate assembly 1500.

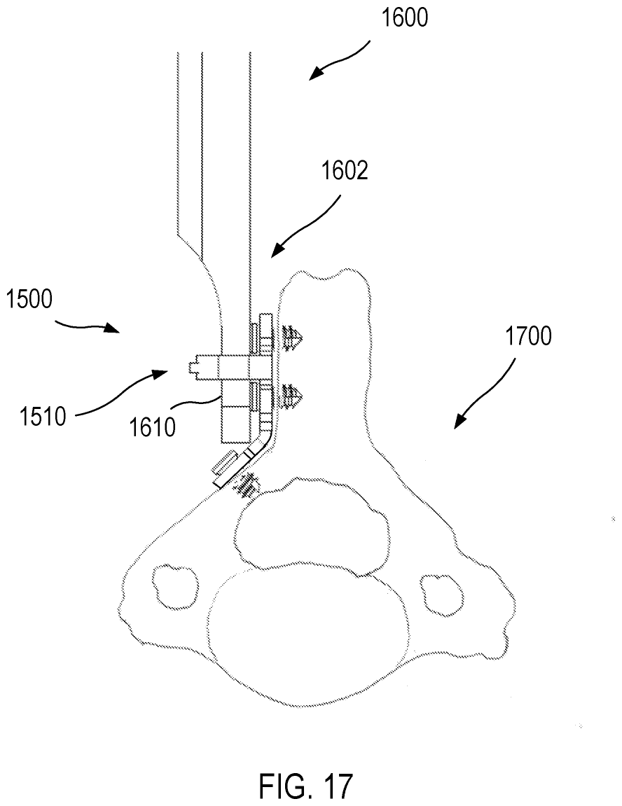

As shown in FIG. 17, plate assembly 1500 can be engaged with or manipulate a vertebra or vertebral body 1700 using bender mechanism 1600. The bender mechanism 1600 can be used to apply a bending force to one or more portions of a loop or ring formed by the elongate members (e.g. to interior surface 1544 shown in FIG. 15F). Bender mechanism 1600 includes a distal section 102 having a shaft portion 1610 that is sized or configured to fit in the loop or ring created by elongate arms of an implant plate 1510 of the plate assembly 1500.

An implant plate can be converted from a first configuration (e.g. flat) to a second configuration (e.g. bent) by deforming or bending one or more portions of the implant plate. FIGS. 15A, 15B, and 15E depict implant plate 1510 in a flat geometry or configuration, and FIGS. 15C, 15D, 15F, 15G, 16, and 17 depict implant plate 1510 in a final bent geometry or configuration (e.g. after bending), according to embodiments of the present invention.

According to some embodiments, bender mechanism 1600 may be used as part of an instrument assembly. For example, with reference to instrument assembly 100 depicted in FIG. 10E, bender mechanism 1600 could replace primary instrument 110 and therefore could be used with secondary instrument 120.

Similarly, according to some embodiments, and with reference to instrument assembly 1100 depicted in FIG. 13, bender mechanism 1600 could replace primary instrument 1110 and therefore could be used with secondary instrument 1120.

Although the preceding description contains significant detail in relation to certain preferred embodiments, it should not be construed as limiting the scope of the invention but rather as providing illustrations of the preferred embodiments.

Embodiments of the present invention encompass kits having one or more components of a cervical laminoplasty system as disclosed herein. In some embodiments, the kit includes one or more cervical laminoplasty system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A cervical laminoplasty system, comprising:
a primary instrument having a proximal portion and a distal portion;
an implant plate having a medial side and a lateral side;
one or more medial bone screws configured to secure a medial side of the implant plate to a medial portion of a lamina of a vertebra; and
one or more lateral bone screws configured to secure a lateral side of the implant plate to a lateral portion of a lamina of a vertebra.

2. The system according to claim 1, wherein the proximal portion of the primary instrument comprises a handle.

3. The system according to claim 1, wherein the distal portion of the primary instrument is configured to engage the implant.

4. The system according to claim 1, wherein the distal portion of the primary instrument is configured to engage an implant post of the implant plate assembly.

5. The system according to claim 1, wherein the implant plate comprises an implant post, and wherein the primary instrument is configured to engage the implant post.

6. The system according to claim 1, further comprising a secondary instrument having a proximal portion and a distal portion, wherein the proximal portion is configured to engage the primary instrument, and wherein the distal portion is configured to engage a second lamina of the vertebra.

7. The system according to claim 1, wherein the primary instrument and the secondary instrument are coupled together via a screw.

8. The system according to claim 1, wherein the implant plate of the implant plate assembly comprises a lateral side, a medial side, and a bridge disposed between the lateral side and the medial side.

9. A method of performing a cervical laminoplasty procedure, the method comprising:
creating a partial cut in a first lamina of a vertebra of a patient;
attaching an implant plate with the vertebra of the patient, wherein a lateral side of the plate is attached with a lateral portion of the first lamina of the vertebra, and wherein a medial side of the plate is attached with a medial portion of the first lamina of the vertebra;
creating a full cut in a second lamina of the vertebra of the patient;
engaging a primary instrument of a cervical laminoplasty system with the implant plate; and
moving the first instrument of the cervical laminoplasty system relative to the patient to form a bend in the implant plate, the bend disposed between the lateral side of the implant plate and the medial side of the implant plate.

10. The method according to claim 9, wherein the primary instrument comprises a distal portion and the secondary instrument comprises a distal portion, wherein the step of engaging the primary instrument of the cervical laminoplasty system with the implant plate comprises engaging the distal portion of the primary instrument with the medial side of the implant plate, and wherein the step of engaging the secondary instrument of the cervical laminoplasty system with the full cut comprises engaging the distal portion of the secondary instrument with the full cut.

11. The method according to claim 10, wherein the medial side of the implant plate comprises an implant post, and the step of engaging the distal portion of the primary instrument with the medial side of the implant plate comprises engaging the distal portion of the primary instrument with the implant post of the implant plate.

12. The method according to claim 9, wherein distal portion of the primary instrument comprises a hook, and the step of engaging the distal portion of the primary instrument with the implant post comprises engaging the hook of the distal portion of the primary instrument with the implant post.

13. The method according to claim 9, wherein the step of attaching the implant plate with the vertebra of the patient comprises securing the lateral side of the implant plate with the lateral portion of the first lamina using one or more lateral bone screws.

14. The method according to claim 9, wherein the step of attaching the implant plate with the vertebra of the patient comprises securing the medial side of the implant plate with the medial portion of the first lamina using one or more medial bone screws.

15. The method according to claim 9, wherein the implant plate comprises a bridge disposed between the lateral side of the implant plate and the medical side of the implant plate, and the step of forming the bend in the implant plate comprises bending the bridge.

16. The method according to claim 9, wherein the bend has an angle of about 60 degrees.

17. The method according to claim 9, wherein the bend has an angle with a value within a range from about 1 degree to about 60 degrees.

18. The method according to claim 9, wherein the step of moving the first instrument and the second instrument of the cervical laminoplasty system relative to the patient comprises rotating the first instrument and the second instrument of the cervical laminoplasty system relative to the lateral portion of the first lamina of the vertebra.
